(19) United States
(12) Patent Application Publication
(10) Pub. No.: US 2021/0270835 A1
(43) Pub. Date: Sep. 2, 2021

(54) BIOMARKERS FOR UROTHELIAL CARCINOMA AND APPLICATIONS THEREOF

(71) Applicant: CHINA MEDICAL UNIVERSITY, Taichung (TW)

(72) Inventors: Chao-Jung CHEN, Taichung (TW); Chiu-Ching HUANG, Taipei City (TW)

(73) Assignee: CHINA MEDICAL UNIVERSITY, Taichung (TW)

(21) Appl. No.: 17/253,984

(22) PCT Filed: Jun. 21, 2019
(86) PCT No.: PCT/CN2019/092309

§ 371 Date: Dec. 18, 2020

Related U.S. Application Data
(60) Provisional application No. 62/688,138, filed on Jun. 21, 2018.

(51) Int. Cl. G01N 33/574

(52) U.S. Cl. CPC G01N 33/57407; G01N 2333/9015; G01N 2333/4703; G01N 2333/4739; G01N 2333/4742; G01N 2333/475

(57) ABSTRACT

Disclosed are a biomarker, method and assay kit for identifying and screening for urothelial carcinoma (UC) in a subject in need.

### RELATED APPLICATION

This application claims the benefit of U.S. provisional application number U.S. Ser. No. 62/688,138, filed Jun. 21, 2018 under 35 U.S.C. § 119, the entire content of which is herein incorporated by reference.

### TECHNOLOGY FIELD

The present invention relates to a biomarker, method and assay kit for identifying and screening for urothelial carcinoma (UC) in a subject in need.

### BACKGROUND OF THE INVENTION

Urothelial carcinoma (UC) encompass carcinomas of the bladder, ureters, and renal pelvis and is the ninth most prevalent malignancy worldwide 1. Currently, UC is diagnosed by urine cytology, intravenous or computed tomography urography, and biopsy-aided cystoscopy². There have been studies reporting urinary protein markers for the detection of UC biomarkers¹⁰⁻¹³. Among them, BTA, NMP22, CYFRA21.1 and midikine have been approved as cancer biomarkers by the Food and Drug Administration of the United States of America (FDA). Although urine cytology and urography are noninvasive, their sensitivity and specificity varies with UC location and grade by more than 30%.³⁻⁴ Cystoscopy is currently the most accurate method for the diagnosis of UC; however, it is invasive and expensive⁵. In addition, high prevalence of UC in patients with chronic kidney disease (CKD) have been reported recently¹⁴⁻¹⁵, i.e., high percentage of UC patients also have CKD. Most of the reported UC protein biomarkers are found not sufficiently accurate to distinguish UC patients from CKD patients.

Therefore, there is still a need to provide a biomarker for UC detection.

### SUMMARY OF THE INVENTION

In this present invention, it is unexpectedly found that particular proteins including GARS, BRDT, HDGF and CYBP are specifically and highly expressed in UC patients compared to CKD and healthy normal control groups. These proteins thus can be used as specific biomarkers for UC detection. Therefore, the present invention provides a technique for detecting or screening for UC, using one or more proteins including GARS, BRDT, HDGF and/or CYBP, as biomarkers. In particular, the technique of the present invention can be performed in a non-invasive approach by detecting these biomarkers in urine samples. In addition, the technique of the present invention can effectively distinguish UC patients from not only normal/healthy individuals (without CKD) but also CKD patients. The technique of the present invention can further combine conventional biomarkers known in the art to improve the accuracy of the detection. The technique of the present invention can subsequently combine proper therapy for patients based on the results of detection.

In one aspect, the present invention provides a method for detecting urothelial carcinoma (UC) in a subject, the method comprising:

(i) providing a biological sample obtained from the subject to be tested; and

(ii) detecting a first biomarker in the biological sample to obtain a first detection level, comparing the first detection level with a first reference level for said first biomarker to obtain a first comparison result, and assessing whether the subject has UC or is at risk of developing UC based on the first comparison result, wherein the first biomarker is selected from the group consisting of GARS (Glycine-tRNA ligase or glycyl-tRNA synthetase), BRDT (bromodomain testis-specific protein), HDGF (hepatoma-derived growth factor), CYBP (calcyclin-binding protein), and any combinations thereof, and an increase in the first detection level as compared to the first reference level indicates that the subject has UC or be at risk of developing UC; and optionally

(iii) conducting a second detection, which comprises detecting a second biomarker in the biological sample to obtain a second detection level, comparing the second detection level with a second reference level for said second biomarker to obtain a second comparison result, and assessing whether the subject has UC or is at risk of developing UC based on the second comparison result, wherein the second biomarker is selected from the group consisting of midikine, CYFRA21.1 (cytokeratin 19 fragment 21-1), NUMA1 (NMP22) (nuclear matrix protein number 22), and any combinations thereof, and an increase in the second detection level as compared to the second reference level indicates that the subject has UC or be at risk of developing UC.

In some embodiments, the first biomarker includes GARS.

In some embodiments, the first biomarker includes GARS, in combination with BRDT, HDGF and/or CYBP.

In some embodiments, the detection is carried out by mass spectrometry or immunoassay.

In some embodiments, the biological sample is a urine sample.

In some embodiments, the subject is not a CKD patient.

In some embodiments, the subject is a CKD patient.

In some embodiments, if the subject is determined to have UC, the subject is then subjected to a method of treatment for treating UC.

In some embodiments, the method of the present invention comprises detecting the first biomarker and the second biomarker in the biological sample, wherein the first biomarker includes GARS, in combination with BRDT, HDGF and/or CYBP, and the second biomarker includes midikine, CYFRA21.1 and/or NUMA1 (NMP22). In certain examples, the method of the present invention comprises detecting the first biomarker including GARS, in combination with BRDT, HDGF and/or CYBP, and detecting the second biomarker including midikine, CYFRA21.1 and NUMA1 (NMP22).

In a further aspect, the present invention provides a kit for performing a method as described herein which comprises a first reagent that specifically recognizes the first biomarker, and/or a second reagent that specifically recognizes the second biomarker, and instructions for using the kit to detect the presence or amount of the first biomarker and/or the second biomarker.

Also provided is a use of a reagent that specifically recognizes the biomarker(s) as described herein for detecting UC, or for manufacturing a kit or a composition for detecting UC.

In some embodiments, the reagent is selected from the group consisting of (i) a molecule that specifically recognizes GARS, (ii) a molecule that specifically recognizes BRDT, (iii) a molecule that specifically recognizes HDGF, (iv) a molecule that specifically recognizes CYBP, and (v) any combination of (i) to (iv).

In some embodiments, the reagent further comprises (vi) a molecule that specifically recognizes midikine, (vii) a molecule that specifically recognizes CYFRA21.1, (viii) a molecule that specifically recognizes NUMA1 (NMP22), and/or (ix) any combination of (vi) to (viii).

The details of one or more embodiments of the invention are set forth in the description below. Other features or advantages of the present invention will be apparent from the following detailed description of several embodiments, and also from the appended claims.

### BRIEF DESCRIPTION OF THE DRAWINGS

To illustrate the invention, the embodiments are illustrated in the following. However, it should be understood that the invention is not limited to the preferred embodiments shown.

In the drawings:

FIG. 1 shows the box-plot of GARS, BRDT, HDGF and/or CYBP protein levels in urine samples of normal, CKD and UC subjects.

FIG. 2 shows the AUC values of our discovered 4-biomarker panel (GARS, BRDT, HDGF and/or CYBP).

FIG. 3 shows the ROC curve analysis of Midkine, CYFRA 21 (normal control n=179, CKD control=171, UC=172) and NUMA1 (NMP22) (normal n=101, CKD control=149, UC=150)

FIG. 4 shows the ROC comparison of a published marker panel (Midkine, CYFRA 21.1 and NUMA1) and the novel marker panel of the present invention (GARS, BRDT, HDGF and CYBP) in the discrimination of (upper) UC and CKD, (lower-left) UC and control (normal+CKD) and (lower-right) UC and normal UC.

### DETAILED DESCRIPTION OF THE EMBODIMENTS

In order to provide a clear and ready understanding of the present invention, certain terms are first defined. Additional definitions are set forth throughout the detailed description. Unless defined otherwise, all technical and scientific terms used herein have the same meanings as is commonly understood by one of skill in the art to which this invention belongs.

As used herein, the articles “a” and “an” refer to one or more than one (i.e., at least one) of the grammatical object of the article. By way of example, “an element” means one element or more than one element.

As used herein, the term “about” or “approximately” refers to a degree of acceptable deviation that will be understood by persons of ordinary skill in the art, which may vary to some extent depending on the context in which it is used. In general, “about” or “approximately” may mean a numeric value having a range of ±10% around the cited value.

As used herein, the term “comprise” or “comprising” is generally used in the sense of include/including which means permitting the presence of one or more features, ingredients or components. The term “comprise” or “comprising” encompasses the term “consists” or “consisting of.”

As used herein, the terms “subject,” “individual” and “patient” refer to any mammalian subject for whom diagnosis, prognosis, treatment, or therapy is desired, particularly humans. Other subjects may include cattle, dogs, cats, guinea pigs, rabbits, rats, mice, horses, and so on.

As used herein, the term “nucleic acid fragment,” “nucleic acid” and “polynucleotide,” used interchangeably herein, refer to a polymer composed of nucleotide units, including naturally occurring nucleic acids, such as deoxyribonucleic acid (“DNA”) and ribonucleic acid (“RNA”) as well as nucleic acid analogs including those which have non-naturally occurring nucleotides. Thus, these terms include, but are not limited to, single-, double-, or multi-stranded DNA or RNA, genomic DNA, cDNA, mRNA, DNA-RNA hybrids, or a polymer comprising purine and pyrimidine bases or other natural, chemically or biochemically modified, non-natural, or derivatized nucleotide bases. It will be understood that when a nucleic acid fragment is represented by a DNA sequence (i.e., A, T, G, C), this also includes an RNA sequence (i.e., A, U, G, C) in which “U” replaces “T.”

As used herein, the term “primer” as used herein refers to a specific oligonucleotide sequence which is complementary to a target nucleotide sequence and used to hybridize to the target nucleotide sequence. A primer serves as an initiation point for nucleotide polymerization catalyzed by either DNA polymerase, RNA polymerase or reverse transcriptase. For example, primers for GARS, BRDT, HDGF, CYBP, midikine, CYFRA21.1 and NUMA1 (NMP22) as used herein, respectively, are those which are capable to hybridize to the nucleotide sequence of the individual target genes to initiate nucleotide polymerization and produce the nucleotide products as expected based on the design of the sequences of the primers.

As used herein, the term “probe” as used herein refers to a defined nucleic acid segment (or nucleotide analog segment, e.g., polynucleotide as defined herein) which can be used to identify a specific polynucleotide sequence present in samples during hybridization, said nucleic acid segment comprising a nucleotide sequence complementary of the specific polynucleotide sequence to be identified. Typically, a probe can produce a detectable signal since it is labeled in some way, for example, by incorporation of a reporter molecule such as a fluorophore or radionuclide or an enzyme. For example, probes for GARS, BRDT, HDGF, CYBP, midikine, CYFRA21.1 and NUMA1 (NMP22), as used herein, respectively, are those which are capable to specifically hybridize to the corresponding nucleotide sequence of the individual target genes and produce detectable signals caused by such hybridization.

As used herein, the term “hybridization” as used herein shall include any process by which a strand of nucleic acid joins with a complementary strand through base pairing. Relevant technologies are well known in the art and described in, for example, Sambrook et al., Molecular Cloning: A Laboratory Manual, 2^{nd} ed., Cold Spring Harbor Laboratory Press (1989), and Frederick M. A. et al., Current Protocols in Molecular Biology, John Wiley & Sons, Inc. (2001). Typically, stringent conditions are selected to be about 5 to 30° C. lower than the thermal melting point (Tₘ) for the specified sequence at a defined ionic strength and pH. More typically, stringent conditions are selected to be about 5 to 15° C. lower than the Tₘ for the specified sequence at a defined ionic strength and pH. For example, stringent hybridization conditions will be those in which the salt concentration is less than about 1.0 M sodium (or other salts) ion, typically about 0.01 to about 1 M sodium ion concentration at about pH 7.0 to about pH 8.3 and the temperature is at least about 25° C. for short probes (e.g., 10 to 50 nucleotides) and at least about 55° C. for long probes (e.g., greater than 50 nucleotides). An exemplary non-stringent or low stringency condition for a long probe (e.g., greater than 50 nucleotides) would comprise a buffer of 20 mM Tris, pH 8.5, 50 mM KCl, and 2 mM MgCl₂, and a reaction temperature of 25° C.

As used herein, the term “encode” as used herein refers to the inherent property of specific sequences of nucleotides in a polynucleotide (e.g., a gene, a cDNA, or an mRNA) to serve as templates for synthesis of a gene product having either a defined sequence of nucleotides (i.e., rRNA, tRNA and mRNA) or a defined sequence of amino acids and the biological properties resulting therefrom.

As used herein, the term “expression” as used herein refers to the realization of genetic information encoded in a gene to produce a gene product such as an unspliced RNA, an mRNA, a splice variant mRNA, a polypeptide or protein, a post-translationally modified polypeptide, a splice variant polypeptide and so on.

As used herein, the term “expression level” refers to the amount of a gene product expressed by a particular gene in cells which can be determined by any suitable method known in the art.

As used herein, the terms “polypeptide” and “protein,” used interchangeably herein, refer to a polymeric form of amino acids of any length, which can include coded and non-coded amino acids, chemically or biochemically modified or derivatized amino acids, and polypeptides having modified peptide backbones.

As used herein, the term “antibody” means an immunoglobulin protein which is capable of binding an antigen. Antibody as used herein is meant to include the entire antibody as well as any antibody fragments (e.g., F(ab′).sub.2, Fab′, Fab, Fv) capable of binding the epitope, antigen, or antigenic fragment of interest. Antibodies of the invention are immunoreactive or immunospecific for and therefore specifically and selectively bind to a protein of interest, e.g., GARS, BRDT, HDGF, CYBP, midikine, CYFRA21.1 and NUMA1 (NMP22) proteins. Antibodies for the proteins of interest are preferably immunospecific, i.e., not substantially cross-reactive with related materials, although they may recognize their homologs across species. The term “antibody” encompasses all types of antibodies (e.g., monoclonal and polyclonal).

As used herein, the term “diagnosis” as used herein generally includes determination as to whether a subject is likely affected by a given disease, disorder or dysfunction. The skilled persons often make a diagnosis on the basis of one or more diagnostic indicators, i.e., a marker, the presence, absence, or amount of which is indicative of the presence or absence of the disease, disorder or dysfunction. It will be understood in the art that diagnosis does not mean determining the presence or absence of a particular disease with 100% accuracy, but rather an increased likelihood of the presence of certain disease in a subject.

As used herein, the term “treatment” refers to the application or administration of one or more active agents to a subject afflicted with a disorder, a symptom or condition of the disorder, or a progression of the disorder, with the purpose to cure, heal, relieve, alleviate, alter, remedy, ameliorate, improve, or affect the disorder, the symptom or condition of the disorder, the disabilities induced by the disorder, or the progression or predisposition of the disorder.

As used herein, the term “urothelial carcinoma” or “UC” refer to cancer in the lining of the urinary tract, including carcinomas of the bladder, ureters, and renal pelvis.

As used hereon, the term “chronic kidney disease (CKD)” as used herein refers to progressive loss of kidney function over time typically months or even years. Exemplary symptoms may include, but are not limited to, hyperphosphatemia (i.e., for example, >4.6 mg/dl) or low glomerular filtration rates (i.e., for example, <90 ml/minute per 1.73 m² of body surface). In some cases, a patient is diagnosed with chronic kidney disease wherein such patient has i) a sustained reduction in GFR<60 ml/min per 1.73 m² of body surface for 3 or more months; or ii) a structural or functional abnormality of renal function for 3 or more months even in the absence of a reduced GFR. Structural or anatomical abnormalities of the kidney may include persistent microalbuminuria or proteinuria or hematuria or presence of renal cysts.

As used herein, the term “a normal individual” may be used to refer to an individual who is basically in a healthy condition without particular diseases (e.g., UC, CKD), and may refer to a single normal/healthy individual or a group of normal/healthy individuals.

As used herein, the term “a control individual” may be used to refer to an individual who does not suffer from a disease of interest (e.g., UC), and may refer to a single control individual or a group of control individuals. In some embodiments, a control individual may refer to a normal/healthy individual, or a CKD patient without UC, or both. In some embodiments, a control individual may refer to a population including normal/healthy individuals and CKD patients without UC.

As used herein, an “aberrant amount” means an amount of an indicator that is increased as compared to the amount in a subject free from a target disease (e.g., UC) or a reference amount. Specifically, for example, an aberrant amount can be higher than a reference amount by more than 5%, 10%, 20%, 30%, 40%, 50%, 60%, 70%, 80%, 90% or 100% or more. A reference amount can refer to the amount measured in control individuals. In this art, a range of values of control amounts can be obtained by analyzing detected amounts of a marker in samples from a population of control individuals using conventional detection and statistic methods.

As used herein, “low expression” and “high expression” for a biomarker as used herein are relative terms that refer to the level of the biomarker found in a sample. In some embodiments, low and high expression can be determined by comparison of the biomarker expression level in a control, non-diseased sample, where low expression can refer to a lower or comparable expression level to the expression level in a control, non-diseased sample, and high expression can refer to a higher expression level to the expression level in a control, non-diseased sample.

As used herein, a biological marker (biomarker) is a characteristic (e.g. a protein, an amino acid, a metabolite, gene or genetic expression) that is objectively measured and evaluated as an indicator of normal or abnormal biologic processes, diseases, pathogenic processes, or responses to treatment or therapeutic interventions. Biomarkers can include presence or absence of characteristics or patterns or collections of the characteristics which are indicative of particular biological processes. The biomarker measurement can increase or decrease to indicate a certain biological event or process. A marker is primarily used for diagnostic and prognostic purposes. However, it may be used for therapeutic, monitoring, drug screening and other purposes described herein, including evaluation the effectiveness of a therapeutic.

As used herein, a biological sample to be analyzed by any of the methods described herein can be of any type of samples obtained from a subject to be diagnosed. In some embodiments, a biological sample can be a body fluid sample such as a blood sample, a urine sample or an ascetic sample. Typically, a biological sample is a urine sample. In other embodiments, a blood sample can be whole blood or a faction thereof e.g. serum or plasma, heparinized or EDTA treated to avoid blood clotting. Alternatively, the biological sample can be a tissue sample or a biopsy sample.

The present disclosure is based (at least in part) on the identification of one or more gene products as novel UC biomarkers, including GARS, BRDT, HDGF and/or CYBP. As demonstrated in the examples below, an increased level of these markers is found in the urine samples of individuals suffering from UC. In other words, an increase level of GARS, BRDT, HDGF and/or CYBP is found to be associated with the appearance of UC. Thus, the UC detection method described herein can identify whether an individual has, is suspected of having, or is at the risk of developing UC. The detection method described herein can be applied to any subject, including a patient with CKD or an individual without CKD. The detection method described herein may be workable as an initial, regular and routine (or early-stage) screening method to identify those with UC or at the risk for progressing UC.

Biomarkers for UC as described herein are described as follows.

As used herein, the term “GARS” refers to a glycine tRNA ligase (or glycyl-tRNA synthetase) (GARS) gene product. The GARS protein is known as an enzyme that catalyzes the ligation of glycine to the 3-end of its cognate tRNA. The term “BRDT” refers to a bromodomain testis-specific protein (BRDT) gene product. The BRDT protein is a class of tumour-associated antigens (TAAs) which is generally upregulated in response to DNA hypomethylation, a common feature of cancer cells. The term “HDGF” refers to a hepatoma derived growth factor (HDGF) gene product. The HDGF protein is a heparin binding growth factor which was originally identified from conditioned media of human hepatoma cell line, Huh-7.³⁸⁻³⁹ HDGF overexpression has been reported to be correlated with poorer clinical outcomes of oral cancer⁴⁰, gallbladder cancer⁴¹, lung cancer⁴² and hepatocellular carcinoma⁴³. The term “CYBP” refers to a calcyclin-binding protein (CacyBP) gene product. The CYBP protein was known to inhibit growth in gastric cancer⁴⁴ and renal cell carcinoma⁴⁵ and was also associated with clinical progression in breast cancer⁴⁶ has been patented as a biomarker for lung cancer. The term “midkine” refers to a midkine (MK) gene product. The midkine protein also known as neurite growth-promoting factor 2 (NEGF2), appears to enhance the angiogenic and proliferative activities of cancer cells²¹. Midkine are known to be produced in endothelial cells, fetal astrocytes, renal proximal tubule epithelial cells and Wilms' tumor (kidney) cells. Midkine has been reported as potential marker of non-small cell lung cancer²², bladder cancer²³, head and neck squamous cell carcinoma²⁴ and breast cancer²⁵. The term “CYFRA21.1” refers to a CYFRA21.1 gene product. CYFRA21.1 protein is a cytokeratin-19 fragment and has been well known a FDA approved plasma marker for clinical screening of non-small cell lung cancer 26-27. CYFRA21.1 was also reported as a potential urinary marker of bladder cancer, and its prediction value were widely studied²⁸⁻³⁰. The term “NMP22” refers to a nuclear matrix protein number 22 (NMP22) gene product, The NMP22 protein, also known as NUMA1, associated with mitotic apparatus and has been approved by FDA as a non-invasive urinary biomarker of bladder cancer and commercialized as a rapid screening strip. However, the diagnostic value of bladder cancer using NMP22 is still controversial, which may have sensitivity of 33% to 100% and specificity of 40% to 93%³¹. In our study, NMP22 has the highest AUC value of 0.8 compared to Midkine and CYFRA21.1 in distinguish UC from normal subjects; however, its AUC decreased to 0.64 in distinguish UC from CKD subjects. This result may indicate that NMP22 test for UC may be affected by kidney injury. The amino acid sequences of these protein biomarkers and corresponding nucleotide sequence are well known in the art, for example, GARS: P41250 in UniProt, BRDT: Q58F21 in UniProt, HDGF: P51858 in UniProt, CacyBP: Q9HB71, midkine (MK): P21741 in UniProt, CYFRA21.1: P08727 in UniProt, and NMP22: Q14980 in UniProt.

The presence and amount of the biomarkers as described herein in a biological sample can be determined by routine technology. In some embodiments, the presence and/or amount of the biomarkers as described herein can be determined by mass spectrometry, which allows direct measurements of the analytes with high sensitivity and reproducibility. A number of mass spectrometric methods are available. Examples of mass spectrometry include, but are not limited to, matrix-assisted laser desorption ionization/time of flight (MALDI-TOF), surface-enhanced laser desorption ionisation/time of flight (SELDI-TOF), liquid chromatography-mass spectrometry (LC-MS), liquid chromatography tandem mass spectrometry (LC-MS-MS), and electrospray ionization mass spectrometry (ESI-MS). One certain example of this approach is tandem mass spectrometry (MS/MS), which involves multiple steps of mass selection or analysis, usually separated by some form of fragmentation.

In other embodiments, the presence and/or amount of a biomarker can be determined by an immunoassay. Examples of the immunoassays include, but are not limited to, Western blot, enzyme-linked immunosorbent assay (ELISA), radioimmunoassay (RIA), radioimmunoprecipitation assay (RIPA), immunofluorescence assay (IFA), ELFA (enzyme-linked fluorescent immunoassay), electrochemiluminescence (ECL), and Capillary gel electrophoresis (CGE). In some examples, the presence and/or level of a biomarker can be determined using an agent specifically recognizes said biomarker, such as an antibody that specifically binds to the biomarker.

In other embodiments, the presence and/or amount of a biomarker can be determined by measuring mRNA levels of the one or more genes. Assays based on the use of primers or probes that specifically recognize the nucleotide sequences of the genes as described may be used for the measurement, which include but are not limited to reverse transferase-polymerase chain reaction (RT-PCR) and in situ hybridization (ISH), the procedures of which are well known in the art. Primers or probes can readily be designed and synthesized by one of skill in the art based on the nucleic acid region of interest. It will be appreciated that suitable primers or probes to be used in the invention can be designed using any suitable method in view of the nucleotide sequences of the genes of interest as disclosed in the art.

Antibodies as used herein may be polyclonal or monoclonal. Polyclonal antibodies directed against a particular protein are prepared by injection of a suitable laboratory animal with an effective amount of the peptide or antigenic component, collecting serum from the animal, and isolating specific sera by any of the known immunoadsorbent techniques. Animals which can readily be used for producing polyclonal antibodies as used in the invention include chickens, mice, rabbits, rats, goats, horses and the like.

For the performing of the method described herein, the detection or the measurement of the amount of a biomarker as described herein in the biological sample taken from an individual in need thereof (e.g., a human patient having, suspected of having, or at risk of having UC) is carried out by any method known in the art, such as those described herein, e.g. mass spectrometry or immunoassay. Typically, the biological sample is a urine sample.

In some embodiments, the amount of a biomarker in the sample derived from the candidate individual can be compared to a standard value to determine whether the candidate individual has or is at risk of having UC. The standard value represents the amount of a biomarker as described herein in the control sample. The control sample can be taken from an individual that does not have UC. Additionally, the control sample can be a mixture of samples taken from a group of such individuals. Alternatively, the control individuals are matched to the candidate individual in, for example, age, gender, and/or ethnic background. Preferably, the control sample and the biological sample of the candidate individual are samples of the same species.

In some embodiments, a first group of biomarkers are detected. If a first group of biomarkers i.e. GARS, BRDT, HDGF and/or CYBP is measured to have an amount higher than a control value (e.g., about 10% or more above the control value, a first positive result), the candidate individual may be diagnosed as having, being suspected of having, or at risk of having UC. In some embodiments, a second group of biomarkers are further detected. In addition to a positive result obtained from the first group of biomarkers, if a second group of biomarkers i.e. midikine, CYFRA21.1, NUMA1 (NMP22) is measured to have an amount higher than a control value (e.g., about 10% or more above the control value, a second positive result), the candidate individual may be diagnosed as having, being suspected of having, or at risk of having UC, with increased accuracy.

When an individual, such as a human patient, is diagnosed as having, suspected of having, or at risk of having UC, the individual may undergo further testing (e.g., routine physical testing, including surgical biopsy or imaging methods, such as X-ray imaging, magnetic resonance imaging (MRI), or ultrasound) to confirm the occurrence of the disease and/or to determine the stage and type of UC.

In some embodiments, the methods described herein can further comprise treating the UC patient to at least relieve symptoms associated with the disease. The treatment can be conducted by surgery or administration of conventional medicaments for UC. The medicines can be administered in an effective amount to a subject in need.

As used herein, “effective amount” refers to the amount of each active substance that can be administered to the individual, either alone or in combination with one or more other active substances, to confer therapeutic effect on the individual. The effective amount may vary and must be determined by those skilled in the art, depending on the specific circumstances at the time of administration, the severity of the condition, respective parameters of patients, including age, gender, age, weight, height, physical condition, treatment schedule, the nature of the parallel therapy (if any), the specific route of administration, and other possible factors judged by the knowledge and profession of medical personnel. Such factors are well known to those of ordinary skill in the art and can be introduced without further routine experimentation.

The present invention also provides a kit or composition for performing the method, which comprises a reagent (e.g., an antibody, a primer, a probe, or a labeling reagent) that specifically recognizes a biomarker as described herein. The kit may further comprise instructions for using the kit to detect the presence or amount of the biomarker described herein, thereby detecting UC. The components including the detection reagents as described herein can be packaged together in the form of a kit. For example, the detection reagents can be packaged in separate containers, e.g., a nucleic acid (a primer or a probe) or antibody (either bound to a solid matrix or packaged separately with reagents for binding them to the matrix), a control reagent (positive and/or negative), and/or a detectable label, and the instructions (e.g., written, tape, VCR, CD-ROM, etc.) for performing the assay can also be included in the kit. The assay format of the kit can be a Northern hybridization, a chip or an ELISA, for example. Further provided is use of such reagent for performing a method for predicting UC. Such reagent includes a first reagent that specifically recognizes the first biomarker, and/or a second reagent that specifically recognizes the second biomarker. In some embodiments, such reagent includes a first reagent that is selected from the group consisting of (i) a molecule that specifically recognizes GARS, (ii) a molecule that specifically recognizes BRDT, (iii) a molecule that specifically recognizes HDGF, (iv) a molecule that specifically recognizes CYBP, or (v) any combination of (i) to (iv). In some embodiments, the reagent further comprises (vi) a molecule that specifically recognizes midikine, (vii) a molecule that specifically recognizes CYFRA21.1, (viii) a molecule that specifically recognizes NUMA1 (NMP22), or (ix) any combination of (vi) to (viii). Examples of the reagent can be an antibody, a primer, a probe, or a labeling reagent containing a detectable label (e.g. a fluorescent label) that can specifically recognize a biomarker. The reagent may be mixed with a carrier e.g. a pharmaceutically acceptable carrier to form a composition for the detection or diagnosis purpose. Examples of such carrier include injectable saline, injectable distilled water, an injectable buffer solution and the like

Without further elaboration, it is believed that those skilled in the art will be able to apply the invention to its fullest extent based on the above description. The following specific examples are, therefore, intended to be illustrative, and are not intended to limit the applicable scope of the invention in any way. All documents cited herein are incorporated herein by reference.

### Examples

In the beginning of this project, we reviewed published papers and selected 8 published bladder cancer biomarkers for ELISA validation with our collected samples. Our results showed that only 3 published candidates, midikine, CYFRA21.1, NUMA1 (NMP22) have significant higher expression level in UC cohorts compared to CKD and normal cohorts. The above results may imply that most of published UC biomarkers are associated with CKD diseases and results in low specificity in the distinguish UC patients from CKD patients.

Therefore, we aimed to discover novel and more specific protein biomarkers in urine for early UC diagnosis. Proteomics studies have long been used for protein biomarker discovery and description of various biochemical processes, pathways, and mechanisms in both normal and abnormal physiological states. Urinary proteins are relatively stable under appropriate storage and convenient for collection and diagnosis. In this project, we first used iTRAQ-LC-MS/MS approach to discover potential urinary protein biomarkers to distinguish UC from CKD and healthy cohort. After screening numerous proteins, 27 protein candidates were selected for ELISA validation. Among 27 protein candidates, four proteins GARS, BRDT, HDGF, CYBP were found useful as biomarkers for UC with improved accuracy compared with known UC protein biomarkers (midikine, CYFRA21.1, NUMA1 (NMP22)).

### 1. MATERIALS AND METHODS

1.1 Urine Sample Collection

Urine samples from healthy subjects (n=214), UC patients (n=223) and CKD patients (n=281) were collected. The urine samples were supplied with protein inhibitor and stored at −80° C. before analysis.

1.2 Trypsin Digestion and iTRAQ Labeling

To avoid albumin interference in protein identification, urine samples were first processed with albumin-depletion kit (sigma, PROTBA). For protein digestion, protein samples (50 μg) from each pooling group (healthy, CKD, and UC) were precipitated with acetone and re-dissolved with 20 μl of 25 mM TEAB (triethyl ammonium bicarbonate) buffer (pH 8.5) containing 4M urea. The protein solution was reduced with 0.5 μl of 0.2M Tris(2-carboxyethyl)phosphine hydrochloride (TCEP) for 1 hour at 60° C., followed by alkylation with 1 μl of s-methyl methanethiosulfonate (MMTs) for 10 min at 25° C. in the dark. After diluting the protein sample solution with 60 μl of 25 mM TEAB buffer to reduce the urea concentration to 1M, the protein solution was submitted to trypsin digestion for 12 h at 37° C. (enzyme:substrate ratio of 1:25). To label the tryptic peptide samples, the tryptic-digested peptides (˜50 μg) from each group were then labeled using 35 μl of 114, 115 and 116 tags from 4-plex iTRAQ reagents. After labeling tryptic peptide samples of healthy, CKD and UC groups with mass tags of 114, 115 and 116 of iTRAQ reagents, respectively, the three sample groups were further mixed, followed by off-gel separation and nanoLC-MS/MS analysis.

1.3 Peptide Fractionation by Off Gel Separation

The iTRAQ-labeled peptide mixture was fractionated with an Agilent 3100 OFFGEL fractionator (Agilent Technologies) based on isoelectric focusing (IEF). To focus the peptides based on their isoelectric point, IPG strips with pH 3-10 (GE Healthcar) and a 24-well frame set (Agilent Technologies) were used. The sample solution (diluted to 1440 μl) was loaded into the 24-well frame with 60 μl per well. The strip was focused until 50 kVh was reached with a max voltage of 4500 V, 50 μA, 200 mW settings.

1.4 NanoLC-MS/MS Analysis

NanoLC-MS/MS was performed with a nanoflow UPLC system (UltiMate 3000 RSLCnano system; Dionex, TheNetherlands) coupled with a hybrid Q-TOF mass spectrometer (maXis impact; Bruker). The sample was injected into a tunnel-frit trap column (C18, 5 mm, 100 A°, packed length of 2 cm, 375 mm od. 180 mm id)¹⁶ with a flow rate of 8 ml/min and a duration of 5 min. The trapped analyses were separated by a commercial analytical column (Acclaim PepMap C18, 2 μm, 75 mm×250 mm, Thermo Scientific, USA) with a flow rate of 300 nl/min. An acetonitrile/water gradient of 1%-40% within 90 min was used for peptide separation. For MS/MS detection, peptides with charge 2+, 3+ or 4+ and the intensity greater than 20 counts were selected for data dependent acquisition, which was set to one full MS scan (100-2000 m/z) with 1 Hz and switched to ten product ion scans (100-2000 m/z) with 10 Hz.

1.5 Protein Identification and Quantification

NanoLC-MS/MS spectra were converted to xml files by using DataAnalysis software (version 4.1, Bruker Daltonics). To identify proteins, the mass spectra obtained were compared to those in the SwissProt database by using the MASCOT search algorithm (version 2.3.02). The search parameters for MASCOT included peptide mass tolerance-80 ppm, MS/MS mass tolerance-0.05 Da, taxonomy-human, enzyme-trypsin, fixed modification-methylthio (Cys), variable modification-oxidation (Met), deamidation (Asn/Gln), and iTRAQ4plex (Tyr/Lys/N-term). Peptides were identified if their MASCOT individual ion score was higher than 25. The protein ratio with iTRAQ labeling was processed and calculated by using ProteinScape (version 3.1, Bruker Daltonics).

1.6 Measuring Urine Protein Markers by Enzyme-Linked Immunosorbent Assay (ELISA)

Urine samples were centrifuged at 10000 rpm for 10 min, and the supernatant were stored at −80° C. before analysis. The following protein markers were measured in urine using commercially available enzyme-linked immunosorbent assay (ELISA) kits according to manufacture's manual. BTA: complement factor H-related protein 2 (Cat #CSB-E08926h, cusabio, China); NMP22: NUMA1 (Cat #SEC332Hu, Uscn, China); Midkine (Cat #SEA631Hu, Uscn, China); CYFRA21-1: Keratin, type I cytoskeletal 19 (Cat #SEB246Hu, Uscn, China); TACSTD2: tumor-associated calcium signal transducer 2 (Cat #CSB-EL023072HU, cusabio, China); Blca-1 (Cat #CSB-E14974h, cusabio, China); Blca-4 (Cat #CSB-E14959h, cusabio, China); HAI-1: Kunitz-type protease inhibitor 1 (Cat #CSB-EL022584HU, cusabio, China); HtrA1: serine protease HTRA1 (Cat #SEL604Hu, Uscn, China); BRDT: Human bromodomain testis-specific protein (Cat #CSB-EL002807HU, cusabio, China). GARS: Glycyl tRNA synthetase (Cat #SEC996Hu, Uscn, China). Hepatoma Derived Growth Factor (Cat #SEA624Hu, Uscn, China). Human calcyclin binding protein (Ca #201-12-3361, SunRed, China) Urine creatinine was used to normalize protein concentration.

### 2. RESULTS

2.1 Validate Published Urine Biomarkers of UC and Discover Novel Urine Biomarkers Using Mass Spectrometry

Demographic and clinical characteristics of UC patients and control subgroups of CKD and normal were collected and shown in Table 1. Normal, CKD and UC groups were matched in age. CKD and UC groups were further matched in sex, BMI, creatinine, eGFR and albumin. UC subjects have lower urine creatinine and higher CA125 compared to CKD subjects (Table 1).

TABLE 1
Clinical and biochemical characteristics of normal, CKD and UC
subjects. Data was expressed as mean (SD).

| | | Normal | CKD | UC | |
|---|---|---|---|---|---|
| | Unit | (n = 201) | (n = 281) | (n = 211) | P value |
| Gender | None | 103/98 | 115/79 | 117/80 | # |
| (M/F) | | | | | |
| Age | years | 63.8(2.9) | 62.3 (11.5) | 67.5(10.7) | # |
| BMI | kg/m2 | — | 24.5(4.0) | 24.1(3.8) | # |
| Urine | mgic1L | 158.2 | 56.5(33.7) | 40.3(25.5) | <0.001A,B |
| creatinine | | (142.6) | | | |
| CA125 | Ulml | — | 18.6(44.4) | 16.5(6.0) | # |
| HE4 | pmol/L | — | 267.6 | 371.8 | <0.01B |
| | | | (435.9) | (456.0) | |
| Creatinine | mg/dL | — | 2.21(1.3) | 2.48(2.13) | # |
| eGFR | ml/min | — | 46.1(27.7) | 51.6(31.5) | # |
| Albumin | mg/L | — | 4.0(0.6) | 3.9(0.6) | # |

BMI: Body mass index; eGFR: Estimated glomerular filtration rate; CA125: Carcinoma antigen 125; HE4: Human epididymis protein 4; ^{A}Normal compared with CKD; ^{B}Normal compared with UC; #: No significant difference

To discover novel protein biomarkers for UC, we have used iTRAQ-labeled quantitative proteomics to study urinary proteome in normal, CKD and UC groups. In the triplicates experiments, 2497 proteins were identified with quantification information. There are 175 proteins with protein ratios larger than 1.5 folds in UC/CKD and CKD/normal. The 175 protein candidates were further narrow down by referring 4 published papers¹⁷⁻²⁰, and their protein expression level of UC tissues on Human Atlas website (http://www.proteinatlas.org/). Finally, 27 protein candidates were selected for further ELISA validation (Table 2).

TABLE 2
27 protein candidates for ELISA assay

| | | ELISA validation P value | |
|---|---|---|---|
| Accession | Protein | UC vs CKD (n, n) | CKD vs Nor. (n, n) |
| BRDT | Bromodomain testis-specific protein | ≤0.001 (197, 194) | ≤0.001 (194, 201) |
| KTN1 | Kinectin | 0.038 (116, 92) | 0.021 (92, 35) |
| HRG | Histidine-rich glycoprotein | 0.032 (116, 92) | 0.013 (92, 35) |
| CYBP | Calcyclin-binding protein | ≤0.001 (192, 191) | 0.066 (191, 167) |
| IBP3 | Insulin-like growth factor-binding protein 3 | 0.171 (100, 41) | ≤0.001 (41, 21) |
| GDF8 | Growth/differentiation factor 8 | 0.112 (100, 41) | 0.018 (41, 21) |
| PDS5B | Sister chromatid cohesion protein PDS5 | 0.920 (100, 41) | 0.690 (41, 21) |
| | homolog B | | |
| STK40 | Serine/threonine-protein kinase 40 | 0.296 (100, 41) | 0.933 (41, 21) |
| CCD69 | Coiled-coil domain-containing protein 69 | 0.009 (100, 41) | 0.666 (41, 21) |
| SAP3 | Ganglioside GM2 activator | 0.013 (50, 21) | 0.229 (21, 10) |
| SF3A3 | Splicing factor 3A subunit 3 | 0.60 (50, 21) | 0.882 (21, 10) |
| SKIL | Ski-like protein | 0.148 (50, 21) | 0.057 (21, 10) |
| GPC6 | Glypican-6 | 0.409 (50, 21) | 0.036 (21, 10) |
| CENPF | Centromere protein F | 0.50 (50, 21) | 0.113 (21, 10) |
| MPP8 | M-phase phosphoprotein 8 | 0.265 (50, 21) | 0.123 (21, 10) |
| DECR | 2.4-dienoyl-CoA reductase, mitochondrial | 0.964 (50, 21) | 0.878 (21, 10) |
| FERM2 | Fermitin family homolog 2 | 0.108 (50, 21) | 0.183 (21, 10) |
| TLN1 | Talin-1 | 0.710 (50, 21) | 0.027 (21, 10) |
| OPTN | Optineurin | 0.079 (50, 21) | 0.245 (21, 10) |
| TET2 | Methylcytosine dioxygenase TET2 | 0.095 (50, 21) | 0.170 (21, 10) |
| IBP6 | Insulin-like growth factor-binding protein 6 | 0.295 (50, 21) | 0.145 (21, 10) |
| CAMP2 | Calmodulin-regulated spectrin-associntcd | 0.446 (50, 21) | 0.245 (21, 10) |
| | protein 2 | | |
| GCC2 | GRIP and coiled-coil domain-containing | 0.249 (50, 21) | 0.026 (21, 10) |
| | protein 2 | | |
| TKT | Transketolase | 0.156 (50, 21) | 1.0 (21, 10) |
| HDGF | Hepatoma-derived growth factor | ≤0.001 (209, 214) | ≤0.001 (214, 151) |
| GARS | Glycine-tRNA ligase | ≤0.001 (209, 214) | ≤0.001 (214, 151) |
| STAG1 | Cohesin subunit SA-1 | 0.50 (30, 31) | 0.49 (31, 20) |

Nor.: Normal, n: number of quantified peptides, N: sample number

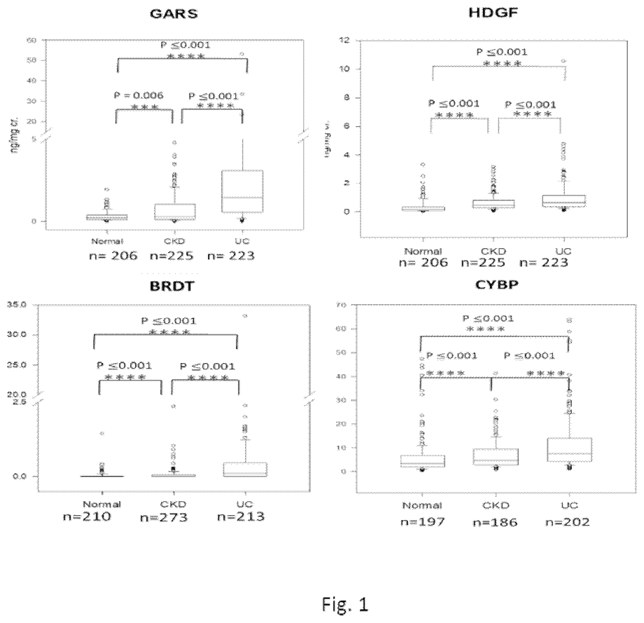

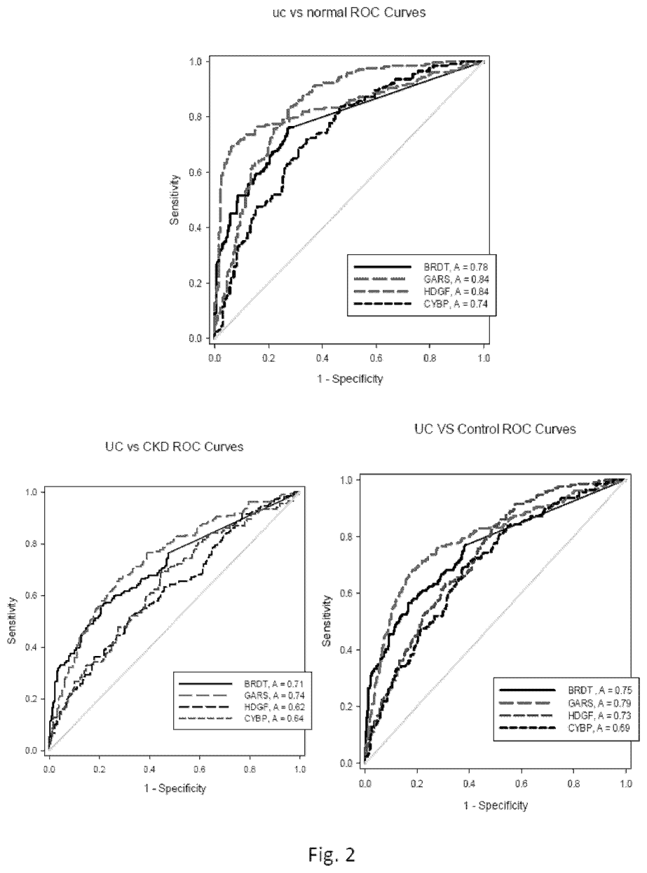

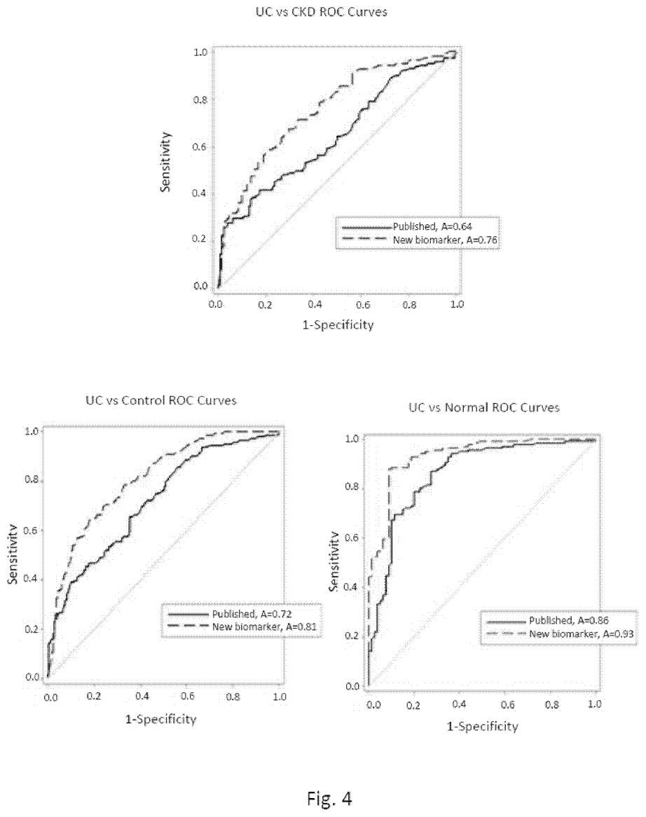

Among 27 protein candidates, BRDT, CYBP, GARS and HDGF were all highly-expressed in a large UC cohort compared to normal and CKD cohort (FIG. 1). The ROC analysis was also performed to investigate whether the levels of the 4 protein markers could distinguish between UC and normal groups, between UC and CKD groups, or between UC and control (normal+CKD) groups. To distinguish between UC and normal, GARS had the highest AUC value of 0.84 when compared to AUC values of HDGF (0.84), BRDT (0.78), and CYBP (0.74). (FIG. 2) After combining the levels of the four markers, the combined AUC value increased to 0.93 (FIG. 4). To distinguish between UC and CKD, GARS had the highest AUC value of 0.74 when compared to AUC values of BRDT (0.71), CYBP (0.64), and HDGF (0.62). After combining the levels of the four markers, the combined AUC value increased to 0.76. To distinguish between UC and control (normal+CKD), GARS still had the highest AUC value of 0.788 when compared to AUC values of BRDT (0.75), HDGF (0.73), and CYBP (0.69). After combining the levels of the four markers, the combined AUC value increased to 0.81.

2.2 ELISA Assay of 8 Published UC Biomarkers in Urine Samples

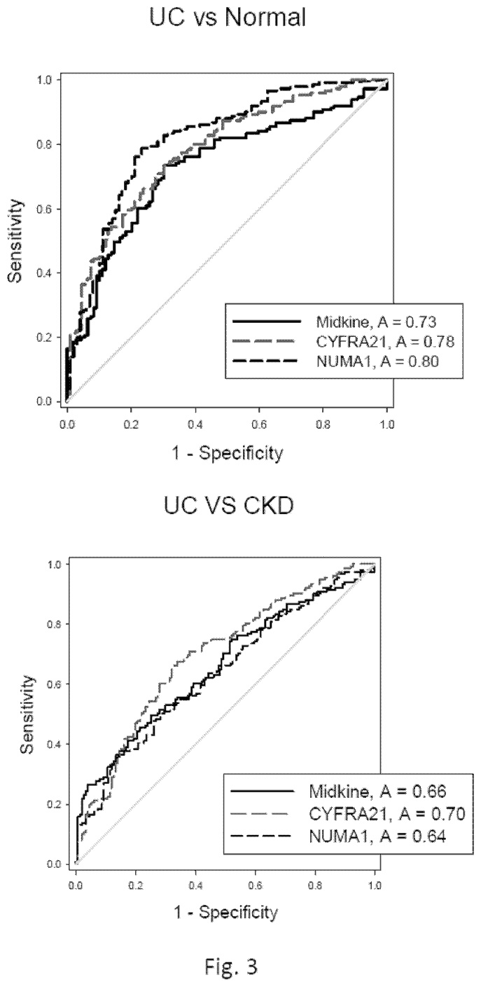

To compare the published protein markers with our discovered GARS, HDGF, BRDT and CYBP markers on Taiwanese UC patients, we first reviewed the articles and selected 8 potential protein markers according to their reported accuracy and validation sample size (Table 3). Among them, BTA, NMP22, CYFRA21.1 and midikine have been approved as cancer biomarkers by FDA. In the verification stage with small sample size of patients (normal: n=20, CKD: n=77, UC: n=89), the expression level of 3 published markers (NUMA1 or NMP22, Midikine, CYFRA21.1) were significant higher in UC patients compared to CKD subjects (p<0.05). In the validation of large sample size (normal: n=179, CKD: n=171, UC: n=172), NUMA1, Midikine, CYFRA21.1 can significantly distinguish UC from normal group with AUC (area under ROC curve) of 0.8, 0.73 and 0.78, respectively; distinguish UC from CKD group with AUC of 0.64, 0.66 and 0.70, respectively (FIG. 3).

TABLE 3
The validation of published UC markers in our urine samples of
UC patients.

| | | Our study | |
|---|---|---|---|
| | | Verification | Validation |
| | | Normal(n = 20), | Normal (n = 179), CKD (n = 171) |
| | | CKD (n = 77), UC (n = 89) | UC (n = 172) |
| Candidates | Reference | P value | P value |
| BTA | A | P = 0.023 (CKD VS N)* | — |
| (FDA approved) | | P = 0.420(CKD VS UC) | |
| BLCA-1 | A | P = 0.002(CKD VS N)*** | — |
| BLCA-4 | | P = 0.379 (CKD VS UC) | |
| NUMA1 = | B | P < 0.001 (CKD VS N)**** | P ≤ 0.001(CKD VS N)**** |
| NMP22 | | P < 0.05 (CKD VS UC)* | P ≤ 0.001(CKD VS UC)**** |
| (FDA approved) | | | |
| CYFRA21.1 | B | P = 0.676(CKD VS N) | P ≤ 0.001(CKD VS N)**** |
| (FDA approved) | | P = 0.003(CKD VS UC)*** | P ≤ 0.001(CKD VS UC)**** |
| TSCSTD2 | C | P = 0.005 (CKD VS N)*** | — |
| | | P = 0.256 (CKD VS UC) | |
| HAI-1 | D | P > 0.05 | — |
| Midikine | D | P = 0.027 (CKD VS N)* | P ≤ 0.001(CKD VS N)**** |
| (FDA approved) | | P = 0.051 (CKD VS UC) | P ≤ 0.001(CKD VS UC)**** |
| HtrA1 | E | P > 0.05 | |

References: A: Urologic Oncology: Seminars and Original Investigations (2013), B:Clin Chim Acta. 2012 Dec 24; 414:93-100., C:J Proteomics. 2012 Jun 27; 75(12):3529-45., D:Br J Cancer. 2013 May 14; 108(9):1854-61., E:Int J Cancer. 2013 Dec 1; 133(11):2650-61.

2.3 ROC Comparison Between the New Marker Panel and the Published Marker Panel

To evaluate the diagnosis performance, the ROC curve of the new four-protein marker panel (GARS, BRDT, HDGF and CYBP) was compared with the published marker panel (BTA, NMP22, CYFRA21.1). FIG. 4 shows that the new 4-protein marker can have higher AUC values when compared to AUC values of the published marker panel in the discrimination between UC and CKD groups (new marker: 0.76 vs. published marker: 0.64), UC and control groups (new marker: 0.81 vs. published marker: 0.72), and UC and normal groups (new marker: 0.93 vs. published marker: 0.86).

2.4 Combination of Multiple Biomarkers to Improve Accuracy of Diagnosis

We combined new four-protein marker panel (GARS, BRDT, HDGF and CYBP) with the published marker panel (midkine, CYFRA21.1 and NUMA1) and found that such combination can have higher AUC values. See Tables 4 and 5.

TABLE 4
ROC curve analysis and resultant AUC values of
multiple biomarkers (UC vs. CKD)

| Biomarkers | AUC values |
|---|---|
| Published candidates (Midkine + CYFRA21.1 + NUMA1) | A = 0.63 |
| New candidates (GARS, BRDT, HDGF and CYBP) | A = 0.76 |
| All Bio-markers (published markers(3) + | A = 0.79 |
| new candidates(4)) | |

TABLE 5
ROC curve analysis and resultant AUC values of
multiple biomarkers (UC vs. normal)

| Biomarkers | AUC values |
|---|---|
| Published candidates (Midkine + CYFRA21.1 + NUMA1) | A = 0.83 |
| New candidates (GARS, BRDT, HDGF and CYBP) | A = 0.91 |
| All Bio-markers (published markers(3) + | A = 0.94 |
| new candidates(4)) | |

### 3. CONCLUSIONS

In conclusion, we have found particular proteins including GARS, BRDT, HDGF and CYBP that are specifically and highly expressed in UC patients compared to CKD and healthy normal control groups. These proteins can be used as biomarkers for UC detection. Specifically, these proteins can be detected in urine samples from patients. Further, these biomarkers can be used to effectively distinguish UC patients from CKD patients. The four-biomarker panel (GARS, BRDT, HDGF and CYBP) has AUC value of 0.81 in distinguish between UC and Control (CKD+normal) and 0.93 in distinguish between UC and normal. The diagnostic value is better than the biomarker panel of published markers of CYFRA21.1, midkine and NUMA1 (NMP-22). Moreover, combination of the four-biomarker panel (GARS, BRDT, HDGF and CYBP) with the published marker panel (midkine, CYFRA21.1 and NUMA1) can have higher AUC values. Therefore, the four biomarkers (GARS, BRDT, HDGF and/or CYBP) are useful for developing a technique for UC detection with improved diagnostic accuracy (better sensitivity and specificity) and in a non-invasive approach.

### REFERENCES

1. Ploeg, M.; Aben, K. K.; Kiemeney, L. A., The present and future burden of urinary bladder cancer in the world. World J Urol 2009, 27 (3), 289-93.
2. Babjuk, M.; Burger, M.; Zigeuner, R.; Shariat, S. F.; van Rhijn, B. W.; Comperat, E.; Sylvester, R. J.; Kaasinen, E.; Bohle, A.; Palou Redorta, J.; Roupret, M.; European Association of, U., EAU guidelines on non-muscle-invasive urothelial carcinoma of the bladder: update 2013. Eur Urol 2013, 64 (4), 639-53.
3. Palou, J.; Rodriguez-Rubio, F.; Huguet, J.; Segarra, J.; Ribal, M. J.; Alcaraz, A.; Villavicencio, H., Multivariate analysis of clinical parameters of synchronous primary superficial bladder cancer and upper urinary tract tumor. The Journal of urology 2005, 174 (3), 859-61; discussion 861.
4. Raitanen, M.-P.; Aine, R.; Rintala, E.; Kallio, J.; Rajala, P.; Juusela, H.; Tammela, T. L.; Group, F., Differences between local and review urinary cytology in diagnosis of bladder cancer. An interobserver multicenter analysis. European urology 2002, 41 (3), 284-289.
5. Lotan, Y; Roehrborn, C. G., Cost-effectiveness of a modified care protocol substituting bladder tumor markers for cystoscopy for the followup of patients with transitional cell carcinoma of the bladder: a decision analytical approach. J Urol 2002, 167 (1), 75-9.
6. Cravatt, B. F.; Simon, G. M.; Yates, J. R., 3rd, The biological impact of mass-spectrometry-based proteomics. Nature 2007, 450 (7172), 991-1000.
7. Niu, L.; Geyer, P. E.; Wewer Albrechtsen, N. J.; Gluud, L. L.; Santos, A.; Doll, S.; Treit, P. V.; Holst, J. J.; Knop, F. K.; Vilsboll, T.; Junker, A.; Sachs, S.; Stemmer, K.; Muller, T. D.; Tschop, M. H.; Hofmann, S. M.; Mann, M., Plasma proteome profiling discovers novel proteins associated with non-alcoholic fatty liver disease. Mol Syst Biol 2019, 15 (3), e8793.
8. Chang, C. T.; Yang, C. Y; Tsai, F. J.; Lin, S. Y; Chen, C. J., Mass Spectrometry-Based Proteomic Study Makes High-Density Lipoprotein a Biomarker for Atherosclerotic Vascular Disease. Biomed Res Int 2015, 2015, 164846.
9. Huang, R.; Chen, Z.; He, L.; He, N.; Xi, Z.; Li, Z.; Deng, Y; Zeng, X., Mass spectrometry-assisted gel-based proteomics in cancer biomarker discovery: approaches and application. Theranostics 2017, 7 (14), 3559-3572.
10. Schiffer, E.; Vlahou, A.; Petrolekas, A.; Stravodimos, K.; Tauber, R.; Geschwend, J. E.; Neuhaus, J.; Stolzenburg, J.-U.; Conaway, M. R.; Mischak, H., Prediction of muscle-invasive bladder cancer using urinary proteomics. Clinical Cancer Research 2009, 15 (15), 4935-4943.
11. Shimwell, N.; Bryan, R.; Wei, W.; James, N.; Cheng, K.; Zeegers, M.; Johnson, P.; Martin, A.; Ward, D., Combined proteome and transcriptome analyses for the discovery of urinary biomarkers for urothelial carcinoma. British journal of cancer 2013, 108 (9), 1854-1861.
12. Yang, N.; Feng, S.; Shedden, K.; Xie, X.; Liu, Y; Rosser, C. J.; Lubman, D. M.; Goodison, S., Urinary glycoprotein biomarker discovery for bladder cancer detection using LC/MS-MS and label-free quantification. Clinical Cancer Research 2011, 17 (10), 3349-3359.
13. Szarvas, T.; Nyirady, P.; Ogawa, O.; Furuya, H.; Rosser, C. J.; Kobayashi, T., Urinary Protein Markers for the Detection and Prognostication of Urothelial Carcinoma. Methods Mol Biol 2018, 1655, 251-273.
14. Chung, C. J.; Huang, C. Y; Tsai, H. B.; Muo, C. H.; Chung, M. C.; Chang, C. H.; Huang, C. C., Sex differences in the development of malignancies among end-stage renal disease patients: a nationwide population-based follow-up study in Taiwan. PLoS One 2012, 7 (9), e44675.
15. Lowrance, W. T.; Ordonez, J.; Udaltsova, N.; Russo, P.; Go, A. S., CKD and the risk of incident cancer. J Am Soc Nephrol 2014, 25 (10), 2327-34.
16. Chen, C. J.; Chen, W. Y; Tseng, M. C.; Chen, Y R., Tunnel frit: a nonmetallic in-capillary frit for nanoflow ultra high-performance liquid chromatography-mass spectrometry applications. Anal Chem 2012, 84 (1), 297-303.
17. Wu, C. C.; Hsu, C. W.; Chen, C. D.; Yu, C. J.; Chang, K. P.; Tai, D. I.; Liu, H. P.; Su, W. H.; Chang, Y S.; Yu, J. S., Candidate serological biomarkers for cancer identified from the secretomes of 23 cancer cell lines and the human protein atlas. Mol Cell Proteomics 2010, 9 (6), 1100-17.
18. Welton, J. L.; Khanna, S.; Giles, P. J.; Brennan, P.; Brewis, I. A.; Staffurth, J.; Mason, M. D.; Clayton, A., Proteomics analysis of bladder cancer exosomes. Mol Cell Proteomics 2010, 9 (6), 1324-38.
19. Chen, C. L.; Lai, Y F.; Tang, P.; Chien, K. Y; Yu, J. S.; Tsai, C. H.; Chen, H. W.; Wu, C. C.; Chung, T.; Hsu, C. W.; Chen, C. D.; Chang, Y S.; Chang, P. L.; Chen, Y T., Comparative and targeted proteomic analyses of urinary microparticles from bladder cancer and hernia patients. J Proteome Res 2012, 11 (12), 5611-29.
20. Chen, C. L.; Lin, T. S.; Tsai, C. H.; Wu, C. C.; Chung, T.; Chien, K. Y; Wu, M.; Chang, Y S.; Yu, J. S.; Chen, Y T., Identification of potential bladder cancer markers in urine by abundant-protein depletion coupled with quantitative proteomics. J Proteomics 2013, 85, 28-43.
21. Kato, M.; Maeta, H.; Kato, S.; Shinozawa, T.; Terada, T., Immunohistochemical and in situ hybridization analyses of midkine expression in thyroid papillary carcinoma. Mod Pathol 2000, 13 (10), 1060-5.
22. Xia, X.; Lu, J. J.; Zhang, S. S.; Su, C. H.; Luo, H. H., Midkine is a serum and urinary biomarker for the detection and prognosis of non-small cell lung cancer. Oncotarget 2016, 7 (52), 87462-87472.
23. Vu Van, D.; Heberling, U.; Wirth, M. P.; Fuessel, S., Validation of the diagnostic utility of urinary midkine for the detection of bladder cancer. Oncol Lett 2016, 12 (5), 3143-3152.
24. Yamashita, T.; Shimada, H.; Tanaka, S.; Araki, K.; Tomifuji, M.; Mizokami, D.; Tanaka, N.; Kamide, D.; Miyagawa, Y; Suzuki, H.; Tanaka, Y; Shiotani, A., Serum midkine as a biomarker for malignancy, prognosis, and chemosensitivity in head and neck squamous cell carcinoma. Cancer Med 2016, 5 (3), 415-25.
25. Li, F.; Tian, P.; Zhang, J.; Kou, C., The clinical and prognostic significance of midkine in breast cancer patients. Tumour Biol 2015, 36 (12), 9789-94.
26. Wieskopf, B.; Demangeat, C.; Purohit, A.; Stenger, R.; Gries, P.; Kreisman, H.; Quoix, E., Cyfra 21-1 as a biologic marker of non-small cell lung cancer. Evaluation of sensitivity, specificity, and prognostic role. Chest 1995, 108 (1), 163-9.
27. Holdenrieder, S.; Wehnl, B.; Hettwer, K.; Simon, K.; Uhlig, S.; Dayyani, F., Carcinoembryonic antigen and cytokeratin-19 fragments for assessment of therapy response in non-small cell lung cancer: a systematic review and meta-analysis. British journal of cancer 2017, 116 (8), 1037-1045.
28. Nisman, B.; Barak, V; Shapiro, A.; Golijanin, D.; Peretz, T.; Pode, D., Evaluation of urine CYFRA 21-1 for the detection of primary and recurrent bladder carcinoma. Cancer 2002, 94 (11), 2914-22.
29. Guo, X. G.; Long, J. J., Cytokeratin-19 fragment in the diagnosis of bladder carcinoma. Tumour biology: the journal of the International Society for Oncodevelopmental Biology and Medicine 2016, 37 (10), 14329-14330.
30. Jeong, S.; Park, Y; Cho, Y; Kim, Y R.; Kim, H. S., Diagnostic values of urine CYFRA21-1, NMP22, UBC, and FDP for the detection of bladder cancer. Clinica chimica acta; international journal of clinical chemistry 2012, 414, 93-100.
31. Mowatt, G.; Zhu, S.; Kilonzo, M.; Boachie, C.; Fraser, C.; Griffiths, T. R.; N'Dow, J.; Nabi, G.; Cook, J.; Vale, L., Systematic review of the clinical effectiveness and cost-effectiveness of photodynamic diagnosis and urine biomarkers (FISH, ImmunoCyt, NMP22) and cytology for the detection and follow-up of bladder cancer. Health Technol Assess 2010, 14 (4), 1-331, iii-iv.
32. Rousseaux, S.; Khochbin, S., New hypotheses for large-scale epigenome alterations in somatic cancer cells: a role for male germ-cell-specific regulators. Epigenomics 2009, 1 (1), 153-61.
33. Shi, J.; Vakoc, C. R., The mechanisms behind the therapeutic activity of BET bromodomain inhibition. Molecular cell 2014, 54 (5), 728-36.
34. Filippakopoulos, P.; Qi, J.; Picaud, S.; Shen, Y; Smith, W. B.; Fedorov, O.; Morse, E. M.; Keates, T.; Hickman, T. T.; Felletar, I.; Philpott, M.; Munro, S.; McKeown, M. R.; Wang, Y; Christie, A. L.; West, N.; Cameron, M. J.; Schwartz, B.; Heightman, T. D.; La Thangue, N.; French, C. A.; Wiest, O.; Kung, A. L.; Knapp, S.; Bradner, J. E., Selective inhibition of BET bromodomains. Nature 2010, 468 (7327), 1067-73.
35. Nicodeme, E.; Jeffrey, K. L.; Schaefer, U.; Beinke, S.; Dewell, S.; Chung, C. W.; Chandwani, R.; Marazzi, I.; Wilson, P.; Coste, H.; White, J.; Kirilovsky, J.; Rice, C. M.; Lora, J. M.; Prinjha, R. K.; Lee, K.; Tarakhovsky, A., Suppression of inflammation by a synthetic histone mimic. Nature 2010, 468 (7327), 1119-23.
36. Andrieu, G.; Belkina, A. C.; Denis, G. V., Clinical trials for BET inhibitors run ahead of the science. Drug discovery today. Technologies 2016, 19, 45-50.
37. Park, M. C.; Kang, T.; Jin, D.; Han, J. M.; Kim, S. B.; Park, Y J.; Cho, K.; Park, Y W.; Guo, M.; He, W.; Yang, X. L.; Schimmel, P.; Kim, S., Secreted human glycyl-tRNA synthetase implicated in defense against ERK-activated tumorigenesis. Proc Natl Acad Sci USA 2012, 109 (11), E640-7.
38. Giri, K.; Pabelick, C. M.; Mukherjee, P.; Prakash, Y S., Hepatoma derived growth factor (HDGF) dynamics in ovarian cancer cells. Apoptosis 2016, 21 (3), 329-39.
39. Nakamura, H.; Izumoto, Y; Kambe, H.; Kuroda, T.; Mori, T.; Kawamura, K.; Yamamoto, H.; Kishimoto, T., Molecular cloning of complementary DNA for a novel human hepatoma-derived growth factor. Its homology with high mobility group-1 protein. J Biol Chem 1994, 269 (40), 25143-9.
40. Lin, Y W.; Li, C. F.; Chen, H. Y; Yen, C. Y; Lin, L. C.; Huang, C. C.; Huang, H. Y; Wu, P. C.; Chen, C. H.; Chen, S. C.; Tai, M. H., The expression and prognostic significance of hepatoma-derived growth factor in oral cancer. Oral Oncol 2012, 48 (7), 629-35.
41. Li, M.; Shen, J.; Wu, X.; Zhang, B.; Zhang, R.; Weng, H.; Ding, Q.; Tan, Z.; Gao, G.; Mu, J.; Yang, J.; Shu, Y; Bao, R.; Ding, Q.; Wu, W.; Cao, Y; Liu, Y, Downregulated expression of hepatoma-derived growth factor (HDGF) reduces gallbladder cancer cell proliferation and invasion. Med Oncol 2013, 30 (2), 587.
42. Ren, H.; Chu, Z.; Mao, L., Antibodies targeting hepatoma-derived growth factor as a novel strategy in treating lung cancer. Mol Cancer Ther 2009, 8 (5), 1106-12.
43. Yoshida, K.; Tomita, Y; Okuda, Y; Yamamoto, S.; Enomoto, H.; Uyama, H.; Ito, H.; Hoshida, Y; Aozasa, K.; Nagano, H.; Sakon, M.; Kawase, I.; Monden, M.; Nakamura, H., Hepatoma-derived growth factor is a novel prognostic factor for hepatocellular carcinoma. Ann Surg Oncol 2006, 13 (2), 159-67.
44. Ning, X. X.; Sun, S. R.; Li, Y; Gong, W. Q.; Liu, L. L.; Sun, L.; Liang, J.; Pan, Y L.; Cheng, Y; Wu, K. C.; Fan, D. M., [The effect of calcyclin binding protein on gastric cancer cell proliferation]. Zhonghua Yi Xue Za Zhi 2006, 86 (46), 3264-8.
45. Sun, S.; Ning, X.; Liu, J.; Liu, L.; Chen, Y; Han, S.; Zhang, Y; Liang, J.; Wu, K.; Fan, D., Overexpressed CacyBP/SIP leads to the suppression of growth in renal cell carcinoma. Biochem Biophys Res Commun 2007, 356 (4), 864-71.
46. Wang, N.; Ma, Q.; Wang, Y; Ma, G.; Zhai, H., CacyBP/SIP expression is involved in the clinical progression of breast cancer. World J Surg 2010, 34 (11), 2545-52.

1. A method for detecting urothelial carcinoma (UC) in a subject, the method comprising:
(i) providing a biological sample obtained from the subject; and
(ii) detecting a first biomarker in the biological sample to obtain a first detection level, comparing the first detection level with a first reference level for said first biomarker to obtain a first comparison result, and assessing whether the subject has UC or is at risk of developing UC based on the first comparison result, wherein the first biomarker comprises at least one selected from the group consisting of GARS, BRDT, HDGF, and CYBP, wherein an increase in the first detection level as compared to the first reference level indicates that the subject has UC or is at risk of developing UC; and
(iii) optionally conducting a second detection, which comprises detecting a second biomarker in the biological sample to obtain a second detection level, comparing the second detection level with a second reference level for said second biomarker to obtain a second comparison result, and assessing whether the subject has UC or is at risk of developing UC based on the second comparison result, wherein the second biomarker comprises at least one selected from the group consisting of midikine, CYFRA21.1, and NUMA1 (NMP22), and an increase in the second detection level as compared to the second reference level indicates that the subject has UC or is at risk of developing UC.

2. The method of claim 1, wherein the first biomarker comprises GARS.

3. The method of claim 1, wherein the first biomarker comprises GARS, in combination with at least one selected from the group consisting of BRDT, HDGF and CYBP.

4. The method of claim 1, wherein the first and optional second detection is carried out by mass spectrometry or immunoassay.

5. The method of claim 1, wherein the biological sample is a urine sample.

6. The method of claim 1, wherein the subject is not a chronic kidney disease (CKD) patient.

7. The method of claim 1, wherein the subject is a chronic kidney disease (CKD) patient.

8. The method of claim 1, further comprising:
treating the subject for UC.

9. The method of claim 1,
wherein the first biomarker and the second biomarker are detected in the biological sample,
wherein the first biomarker comprises GARS, in combination with at least one selected from the group consisting of BRDT, HDGF and CYBP, and
wherein the second biomarker comprises midikine, CYFRA21.1 and optionally NUMA1 (NMP22).

10. The method of claim 9, wherein the first biomarker comprises GARS, BRDT, HDGF and CYBP, and second biomarker comprises midikine, CYFRA21.1 and NUMA1 (NMP22).

11. A kit for performing a method of claim 1, which comprises
a first reagent that specifically recognizes the first biomarker,
optionally a second reagent that specifically recognizes the second biomarker, and
instructions for using the kit to detect the presence or amount of the first biomarker and optionally the second biomarker.

12. The kit of claim 11, wherein the fit reagent is linked to a detectable label.

13. (canceled)

14. (canceled)
